(19) United States
(12) Patent Application Publication
(10) Pub. No.: US 2018/0353069 A1
(43) Pub. Date: Dec. 13, 2018

(54) DISTRIBUTED SLAVE BEACON NETWORK REQUIRING NO MOBILE TELEPHONE OR COMPUTE APPLICATIONS

(71) Applicants: Sameh Sarhan, Santa Clara, CA (US); Mohammad Iman Sadreddin, Union City, CA (US); Lawrence Herbert Zuckerman, Livermore, CA (US); Mariano Ferrario, San Francisco, CA (US)

(72) Inventors: Sameh Sarhan, Santa Clara, CA (US); Mohammad Iman Sadreddin, Union City, CA (US); Lawrence Herbert Zuckerman, Livermore, CA (US); Mariano Ferrario, San Francisco, CA (US)

(73) Assignee: Xtrava Inc, Santa Clara, CA (US)

(21) Appl. No.: 16/005,604

(22) Filed: Jun. 11, 2018

Related U.S. Application Data
(60) Provisional application No. 62/517,944, filed on Jun. 11, 2017.

(51) Int. Cl. A61B 5/00; H04L 29/08; G08B 5/22; G08B 3/10; A61B 5/024; A61B 5/08

(52) U.S. Cl. CPC A61B 5/002; A61B 5/6804; H04L 67/12; A61B 5/08; G08B 3/10; A61B 5/024; G08B 5/22

(57) ABSTRACT

This disclosure describes a means for individuals in random locations, whether or not in motion, to communicate with a wearable wellness monitor or any other device without the need to carry electronic communication equipment, by using a wireless network consisting of short-range nodes distributed throughout a home, clinic or other premises. These nodes provide two-way communication with the individuals using verbal announcements, light signals, audible alarms, voice and word recognition, and hand gestures.

### CROSS-REFERENCE TO RELATED APPLICATION

Priority is claimed under 37 CFR 1.78 and 35 USC 119(e) to U.S. Provisional Application 62/517,944 (XT1705311), filed 11 Jun. 2017, which is incorporated by reference.

### TECHNICAL FIELD

This disclosure relates generally to wearable electronic devices and their wireless communication access methods. This disclosure relates more specifically to a bi-directional wireless access system for wearable electronic devices used for the care of a subject individual or individuals, which is distributed throughout a dwelling or institution, requires neither caregivers nor other interested persons to have cellphones or other common mobile devices on their person, and minimizes disturbance to subjects.

### BACKGROUND

For some years, there have appeared on the market a significant number of health monitoring wearable electronic products that notify caregivers of alarm conditions via Bluetooth protocol, cellular, or WiFi (wireless LAN) networks. The highest power Bluetooth equipment requires too much power drain for wearable equipment battery life, and the lowest power Bluetooth equipment, termed Bluetooth Low Energy (“BLE”) has very limited propagation range—essentially under 30 feet. If the caregiver is too far away from the Subject wearing the health monitoring equipment, alarms will not be detected, and there will be no miscellaneous communications. If WiFi or cellular communication networks are used, such equipment must be located in proximity to the Subject, and the caregiver must carry compatible equipment, such as a Cellular/Smart telephone with him at all times. It has been determined that caregivers and others find it inconvenient to consistently maintain electronic equipment on their person. Moreover, the Subject may be ambulatory and located anywhere within the premises. Therefore, what is needed is a method and system to permit two-way communication between each caregiver or other interested person and each Subject's wearable device without carrying any electronic device, regardless of his location within a residence or clinic premises. Such system should also be capable of providing said communication without disturbing the Subjects, who may, for example, be asleep.

### BRIEF SUMMARY

This Brief Summary is provided as a general introduction to the Disclosure provided by the Detailed Description and Figures, summarizing some aspects of the disclosed invention. It is not a detailed overview of the Disclosure, and should not necessarily be interpreted as identifying key elements of the invention, or otherwise characterizing the scope of the invention disclosed in this Patent Document.

Reference is made to U.S. patent application Ser. No. 15/453,911, (Docket #XT1603101), filed 9 Mar. 2017, which is itself cross referenced to U.S. Provisional Application U.S. 62/306,463. That application contains technical details to further support the instant application and is incorporated with the instant application by reference.

The instant disclosure teaches the use of a multiplicity of specially equipped mesh network beacon nodes that use BLE or any other suitable short range radio or other technology to distribute alarm and other signals to and from one or more monitoring devices throughout premises as described below. The mesh network requires neither WiFi equipment, smart phone or smart phone “applications”, nor standard protocols. For the simplest configurations, overall network virtual connections could be avoided; the monitoring device may simply broadcast or advertise alarms.

Said network provides two-way communication with the Subject's or Subjects' wearable monitoring device(s) and caregivers or other occupants or persons present within the premises. Owing to the specially equipped network beacon nodes, said caregivers or other occupants or persons would have no need to carry phones or other communications equipment nor manual access to such equipment when using said network, but could do so. Such equipment if carried could be configured as de-facto nodes of said mesh network. The wearable health monitoring device automatically sends alarm or other information about a Subject to a nearby node(s), which in turn relays this information to nodes throughout the clinic, home, building, or building unit. Specially equipped network nodes, acting as beacons, relay this alarm or other information to caregivers and/or other occupants who are anywhere in the clinic, building, or building unit via auditory announcements or visible light. Sounds originating from a network node can include annunciators and/or words or phrases. The caregivers can send commands via various spoken phrases, which the receiving node will interpret and send as digital commands that will reach the designated Subject's monitoring device. Subjects who can speak can also send auditory information using key words or phrases to caregivers and the system, such as to call for assistance. Other aspects, features and advantages of the invention will be apparent to those skilled in the art from the following Detailed Description.

### BRIEF DESCRIPTION OF DRAWINGS

For a more complete understanding of this disclosure and its features, reference is now made to the following description, taken in conjunction with the accompanying drawings, in which:

FIG. 1 shows a diagram 100 of a dwelling, institution, or clinical setting. It is intended to illustrate subjects being monitored and the herein described methods of said monitoring to be effectuated throughout the premises and beyond.

FIG. 2 shows a nearby Setting 200 that may include no Subjects and is intended to depict an alternative location for the attendants of the Subjects in FIG. 1.

FIG. 3 shows a Setting 300 similar to that of FIG. 2. However, the FIG. 3 Setting can be anywhere, such as at distances where individuals cannot be attendants of the Subjects.

FIG. 4 shows an example block diagram of a Beacon equipped with the means to operate within a network to permit caregivers and others to receive alarms and other status information from Subjects' monitoring instruments, and send information to same and the network, all without the need to carry electronic equipment.

FIG. 5 shows a Subject wearing a health monitoring module that could include various instruments and circuitry to probe the body for health related data, measure environmental conditions, and provide wireless two-way communication with the outside world.

### DETAILED DESCRIPTION

The various figures, discussed below, and the various embodiments used to describe the principles of the present invention in this patent document are by way of illustration only and should not be construed in any way to limit the scope of the invention. Those skilled in the art will understand that the principles of the invention may be implemented in any type of suitably arranged device or system.

In general, this disclosure provides a means for individuals in random locations, whether or not in motion, to communicate with a wearable wellness monitor or any other device without the need to carry electronic communication equipment, by using a wireless network consisting of specially equipped short-range nodes distributed throughout a home, clinic or other premises. Said wearable wellness monitor or any other device may also be in motion and in a random location. These nodes provide two-way communication with the individuals using verbal announcements, light signals, audible alarms, voice and word recognition, and hand signals. Depending on the implementation, this technique can provide significant benefits in a range of fields, such as surveillance, the monitoring of health or status of equipment, or physical variables.

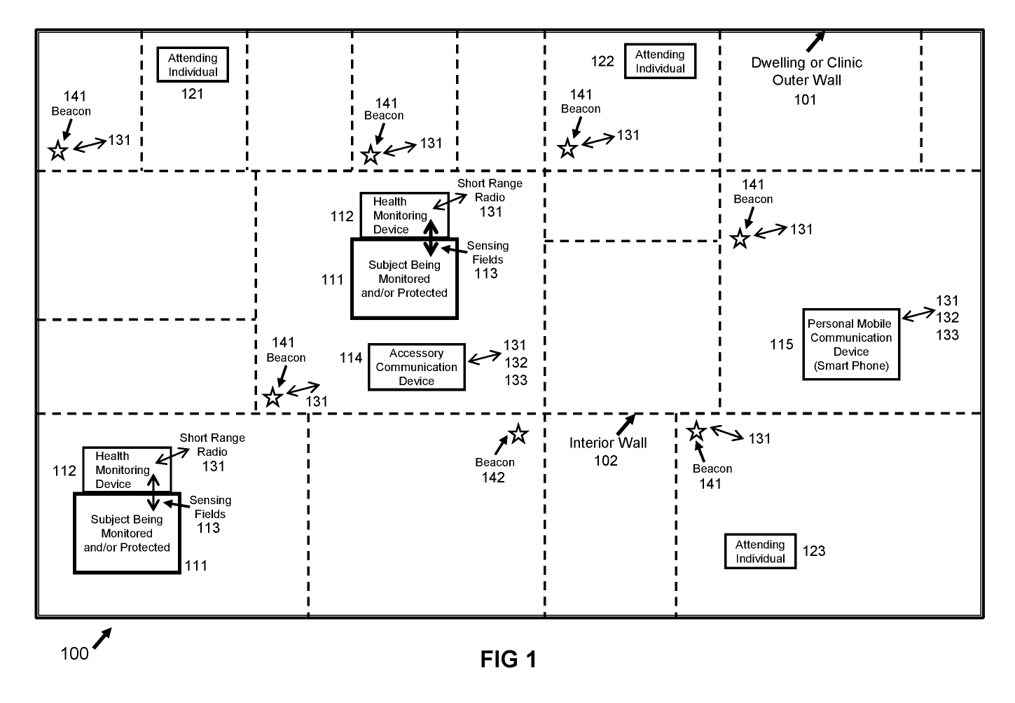

FIG. 1 shows a diagram 100 of a dwelling, institution, or clinical setting, where the outer wall or boundary is depicted by the rectangle 101. The Setting may be divided into a multiplicity of rooms by interior walls 102.

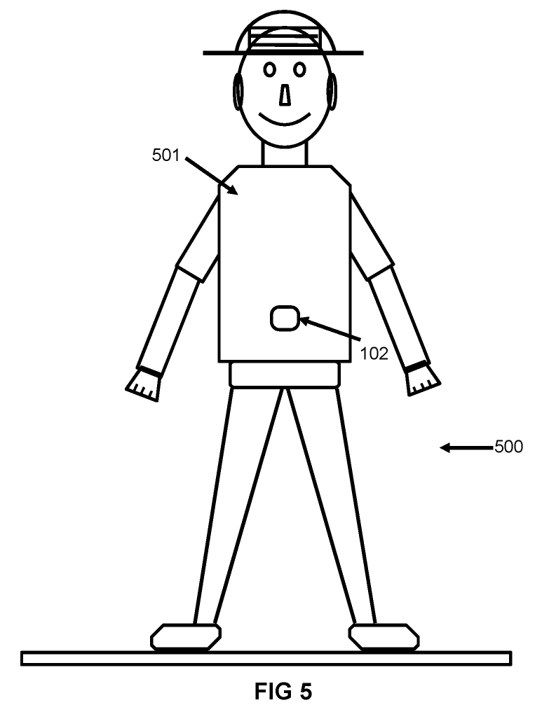

Situated within the Setting may be one or more persons or animals, Subject 111, for example the person shown in FIG. 5, each with a health monitoring device 112 that may or may not be attached to clothing or the like. A particular Subject may be unable to care for himself or call for help, and may be a baby, a child, or infirm adult. Subject 111 may be confined to a bed or cage, lying down or walking around, including throughout the setting.

Health monitoring device 112 may or may not make physical contact with the body of Subject 111. It may be capable of ascertaining a variety of vital signs, for example, heart rate, breathing rate and wetness of clothing. It may be capable of ascertaining other physical aspects such as body position and movement. It may be capable of ascertaining aspects of his immediate physical environment such as light level and/or spectral content, sound, and temperature. The bold double arrow 113 is intended to represent various sensing fields that are used by monitoring device 112.

Health Monitoring Device 112 also includes a short range communications device module (not shown) to transmit collected data and receive programming instructions. The device could be wired or wireless, such as the Low Energy Bluetooth standard (“BLE”). The double arrow 131 is intended to represent radio waves for very short range radio communication.

Accessory Communication Device 114 can communicate with Health Monitoring Device 112 via very short range radio 131, such as BLE. It can also communicate via cellular radio 132, and even WiFi 133 if available. It therefore can be used to transmit data from Device 112 to Attending Individuals 121 who are carrying smart mobile phones, or interested parties located throughout the world, including but not limited to via the “cloud”, and receive programming from same.

Attending Individuals 121, of any quantity, may be caregivers of any kind, for example parents, nurses, or scientists. Such individuals may or may not have smart phones 115 or any electronic communication devices with them. Nevertheless, they may they may want to receive alarms pertaining to the conditions of Subjects 111 and communicate rudimentary information to Devices 112, even if they do not have smart phones 115 or any other electronic communication devices with them. Beacon nodes 141 (“Beacons”), using very short range radio such as BLE, or hard-wired, are equipped to permit this critical communication.

Beacon nodes 141 could communicate directly with one another to form a mesh network and with health monitoring devices 112 via short range radio fields 131. They could also communicate via short range radio fields with accessory communication device 114 or with personal mobile communication device 115. A personal mobile communication device could also be programmed to function as a Beacon.

Beacons 141 could also be hard-wired to other Beacons in addition to having wireless means. Similar Beacons 142 having no wireless means may be hard-wired to other Beacons. All beacons servicing Health Monitoring Devices 112 must have wireless means.

Although Subjects 111 are shown in specific locations, they could be almost anywhere within the Setting and even be in motion, and their health monitoring devices 112 could still communicate with personnel via the network. The local Beacon 141 could also be used to provide the Subjects' locations. Subjects and their health monitoring equipment could not depend, however, on beacons such as 142 that have no functioning wireless module.

Beacons 141, 142 could send auditory or visible signals to attending individuals 121, 122, 123. Such signals could pertain to the status of Subjects 111. Visible signals, such as flashing lights could be used to minimize disturbance to Subjects 111. Beacons 141, 142 could also include motion detectors and video processing to detect the presence of people or hand gestures.

Beacons 141, 142 equipped with voice recognition technology, could receive information from attending individuals 121 etc. in the form of short voiced words or phrases. Such information could include commands to vary the operating modes of health monitoring devices 112 or overall network configuration, register location of ambulatory Subjects 111 or other attending individuals, or announce emergency conditions. Information could also include a caregiver's order to restrict audible announcements from the beacon nodes in his vicinity in order to minimize disturbance to others. The voice recognition technology could be sophisticated enough to distinguish between phrases present in normal conversation and intended communication for the Beacon system. Such capability could reduce the frequency of spurious communication packets.

Personnel could also communicate with a Beacon 141, 142 using flashlight signals, hand clapping, or even room light switch activations.

Each beacon 141, 142 could also be used to gather and communicate environmental and surveillance information pertaining to its immediate location. Such information could include, for instance, whether lighting is on or off, ambient temperature, humidity, smoke, chemical fumes, and unusual sounds. It could also include presence of subjects 111, attending individuals 121, 122, 123, interested persons, or intruders.

Use of ultra-low power radio technology such as BLE, as opposed to WiFi or Cellular, tends to enhance communications security; as there is very little leakage of RF energy outside of the Setting 101.

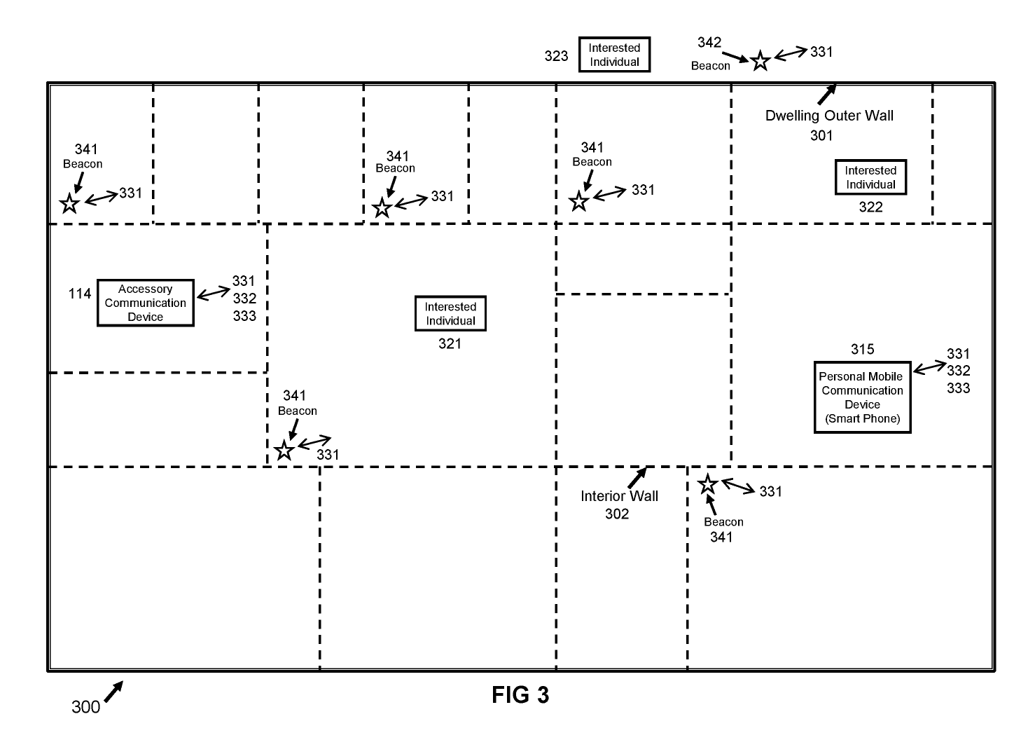

Moreover, BLE was designed for insufficient information bandwidth to communicate real time voice conversation; so personnel who are not in close proximity with health monitoring devices 112 need not fear eavesdropping. However, in order to memorialize abuse to subjects 111 and other inappropriate behavior in their immediate vicinity, devices 112 may be equipped with microphones and a means to store digitized clips of speech and other sounds, along with time stamps. Such sounds could come from subjects 111 or others. This information could be stored in device 112 memory for later gradual downloading via BLE to, for example, accessory communication device 114, for furtherance to interested parties as defined for FIG. 3.

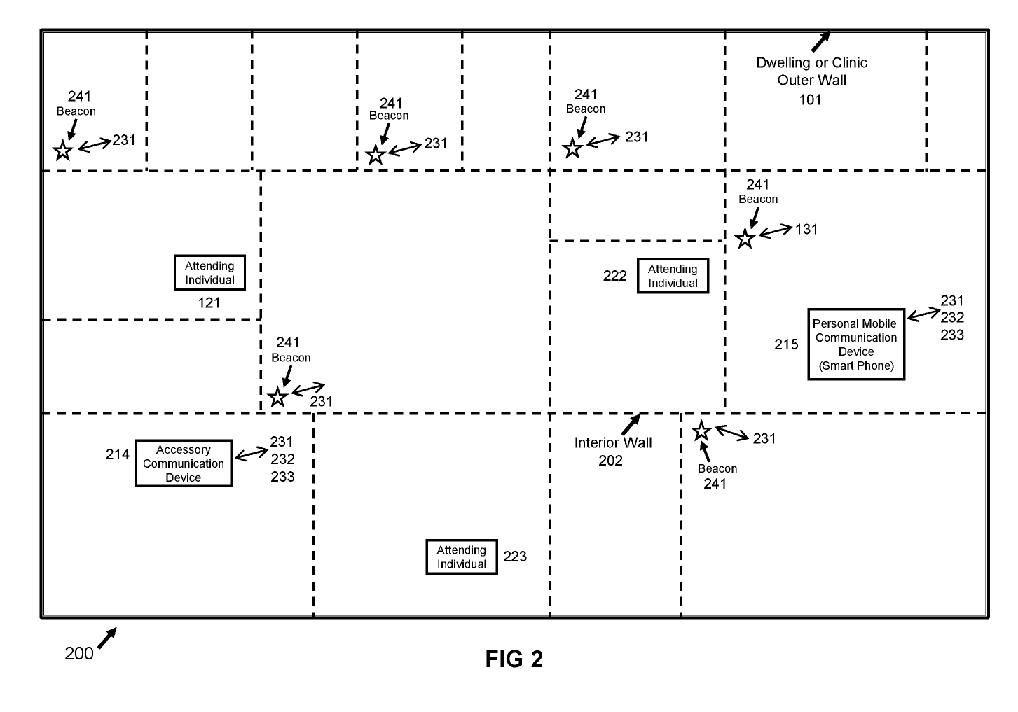

FIG. 2 shows a Setting 200 that appears similar to that of FIG. 1. This Setting may include no Subjects and is intended to depict an alternative location for the attendants of FIG. 1 Subjects, such as Attending Individual 121 or different attendants of the same Subjects, such as Attending Individuals 222 and 223.

The location of the FIG. 2 Setting could be a different clinic within the same hospital or similar institution building, or a nearby building. Said location could be close enough to that of the FIG. 1 setting to allow attending individuals to quickly attend to the Subjects if a Beacon 241 indicates an anomaly, but far enough away or sufficiently shielded to prevent the use of short range radio signals 131, 231 from making the connection.

The connection between the two said Settings could be achieved by any suitable means, such as a dedicated telephone line, using accessory communications devices 114, 214, or personal mobile communication devices 115, 215, or some combination thereof.

FIG. 3 shows a Setting 300 similar to that of FIG. 2. However, the FIG. 3 Setting can be anywhere, such as at distances where individuals cannot be attendants of the Subjects. Thus 321, 322, 323 are each labeled “Interested Individual”, who could for example be relatives of Subject 111. FIG. 3 Setting could be a dwelling, where for example interested individual 323 could be outside but close enough for Beacon 342 to function within the very low power mesh network previously described.

It is anticipated that most Beacon nodes will be placed at fixed locations and powered with mains electricity, with or without backup batteries. However, some Beacon nodes in FIGS. 1, 2, may be strictly battery powered or configured for portable operation; so that for instance they could be deployed outside, such as Beacon 342.

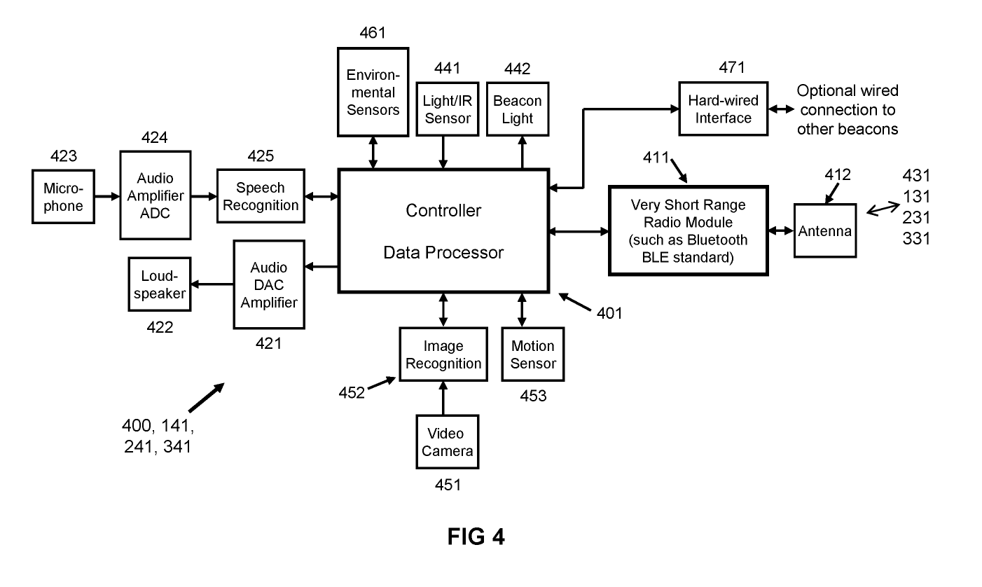

FIG. 4 shows an example block diagram of a Beacon 400, 141, 142, 241, 341, equipped with the means to operate within a network to permit caregivers and others to receive alarms and other status information from the Subjects' 111 monitoring instruments 112, and send information to same and the network, all without the need to carry electronic equipment.

The double arrow near antenna 412 is intended to indicate radio energy 431, 131, 231, 331 propagating to and from other beacon nodes 141, 241, 341 and/or monitoring devices 112. In addition to network-ancillary functions within Controller 401, radio module 411, antenna 412, and hard-wired interface 471, all facilities needed to implement the primary and secondary functions of the instant disclosure are shown.

Beacon Light 442, in lieu of audible alarms that may disturb sleeping subjects or others, could flash in designated patterns to indicate a variety of alarm conditions, for example pulse rate too high or too low, soiled diaper, or overheated machinery. When permissible, or necessary to receive more intricate information, Loudspeaker 422 could be used.

Microphone 423 and Audio Amplifier 424 feed Speech Recognition module 425 to recognize voice commands of personnel and even identify them. Motion Sensor 453 and/or Video Camera 451 with Image Recognition 452 could be used to identify personnel, and receive hand gestures, which could eliminate the need for audible commands such as speech or hand clapping.

Light Sensor 441 could be used to receive commands from personnel by signaling with a visible light or infra-red flashlight, which could be mounted on the wall near every beacon node 400. Crude signaling could be effected even using a room or area light switch.

Environmental Sensors Block 461 could include items that reduce redundancy and cost to maintain a dwelling or other premises by including them as secondary functions of Beacon 400. Sensors therein could be used to gather environmental and surveillance information pertaining to the immediate location. Such information could include, for instance, whether lighting is on or off, ambient temperature, humidity, smoke, chemical fumes, and unusual sounds. It could also include presence of subjects 111, attending individuals 121, 122, 123, interested persons, or intruders.

FIG. 5 shows a Subject 501 wearing an example non-invasive health monitoring module 502, attached to the outside of clothing, that contains various instruments to probe the body. Said module determines health factors such as heart rate, respiration features, clothing wetness and other status, as well as environmental conditions. Said module could also contain one or more wireless devices for two-way communication with the outside world. While this figure shows an adult, said module could be attached to persons of any age, or a wide variety of living organisms. It can be worn on the outside of various regions of the body. Monitoring devices having appropriate functions could also be attached and/or connected to a wide variety of systems and equipment to monitor status or various physical variables.

The details provided in the above description describe particular implementations of the systems for performing the measurements described. Other embodiments could be implemented in any other suitable manner. For example, particular voltages, frequencies, noise levels, gains, resistances, capacitances, and other values may be described. These values are for illustration only. It may be advantageous to set forth definitions of certain words and phrases used throughout this patent document. The term “couple” and its derivatives refer to any direct or indirect communication between two or more elements, whether or not those elements are in physical contact with one another. The terms “transmit,” “receive,” and “communicate,” as well as derivatives thereof, encompass both direct and indirect communication. The terms “include” and “comprise,” as well as derivatives thereof, mean inclusion without limitation. The term “or” is inclusive, meaning and/or. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, have a relationship to or with, or the like.

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

What is claimed is:

1. A method and system to permit individuals who may be anywhere within a premises and who are carrying no electronic communications equipment, to maintain two-way communication with monitoring devices, instruments, or equipment that also may be anywhere within a premises, said system comprising
a multiplicity of network nodes distributed within a premises,
where at least one node maintains wired or wireless connection with said monitoring devices, instruments, or equipment,
where nodes are equipped with means to send information to and receive information from said individuals that does not require the use of electronic equipment on their person.

2. The method and system of claim 1 whereby said monitoring device or instrument is a health monitoring instrument attached to a person or animal being monitored.
